(19) United States
(12) Patent Application Publication
(10) Pub. No.: US 2012/0330367 A1
(43) Pub. Date: Dec. 27, 2012

(54) Orthopedic Check and Balance System

(75) Inventors: Martin Roche, (US); Marc Boillot, Plantation, FL (US); Jason McIntosh, Sugar Hill, GA (US); Carlos Gil, Hallandale Beach, FL (US)

(73) Assignee: Orthosensor, Inc., Sunrise, FL (US)

(21) Appl. No.: 13/527,010

(22) Filed: Jun. 19, 2012

Related U.S. Application Data
(60) Provisional application No. 61/498,647, filed on Jun. 20, 2011.

(51) Int. Cl. A61B 17/56

(52) U.S. Cl. 606/86 R

(57) ABSTRACT

A configurable check and balance system is provided to assess and report orthopedic measurements, including bone cut angles, trial inserts, extension gaps and prosthetic fit. The system can be configured for cut-check, trial-check, alignment and balance, dynamic distraction, and prosthetic trial fit. The measurements can be provided with respect to an anatomical coordinate system defined according to a positioning of a sensorized mechanical plate with respect to one or more referenced anatomical landmarks. In one example, the cut-check provides measurement of varus/valgus angle and anterior/posterior slope for distal femur cuts and proximal tibia cuts. The cut-check permits a surgeon to check bone cuts made by mechanical jigs, guides or patient specific implants (PSI). It also provides distance measurements. Other embodiments are also disclosed.

### CROSS-REFERENCE TO RELATED APPLICATION

This application claims the priority benefit of U.S. Provisional Patent Application No. 61/498,647 filed on 20 Jun. 2011, the entire contents of which are hereby incorporated by reference.

### BACKGROUND

1. Field

The present disclosure relates generally to orthopedic medical devices, and more specifically to surgical electronics for orthopedic instrumentation and measurement.

2. Introduction

During total knee replacement surgery bone cuts are made on the femur and tibia to result in proper alignment and balance. The alignment ensures proper balance and straightness of the leg. The bone cuts can be made with use of mechanical guides and jigs, and more recently, by way of patient specific instruments (PSI). The instruments are attached to the bone and guide the bone saw for the individual bone cuts.

A need remains however for assessing the overall bone cuts, prior to, and when joined and fitted with prosthetics.

### BRIEF DESCRIPTION OF THE DRAWINGS

FIG. 1A depicts an orthopedic cut-check system with GUI in accordance with one embodiment;

FIG. 1B depicts exemplary components of the cut-check system in accordance with one embodiment;

FIG. 1C is an exemplary method for orthopedic cut-check in accordance with one embodiment;

FIG. 1D is an exemplary method of orthopedic cut-check for knee surgery in accordance with one embodiment;

FIG. 1E depicts an illustration for orthopedic cut-check in accordance with one embodiment;

FIG. 1F depicts an illustration for orthopedic cut-check with slotted cutting jigs in accordance with one embodiment;

FIG. 1G depicts a multiple view illustration of an Anatomical Coordinate System created with cut-check in accordance with one embodiment;

FIG. 1H depicts a coronal projection of the Anatomical Coordinate System created with cut-check in accordance with one embodiment;

FIG. 1I depicts a sagittal projection of the Anatomical Coordinate System created with cut-check in accordance with one embodiment;

FIG. 2A depicts an orthopedic trial-check system with GUI in accordance with one embodiment; and

FIG. 2B is an exemplary method for orthopedic trial-check in accordance with one embodiment;

FIG. 2C depicts an illustration for orthopedic trial-check in accordance with one embodiment;

FIG. 3A depicts an orthopedic alignment and balance system in accordance with one embodiment;

FIG. 3B is an exemplary method for orthopedic alignment and balance in accordance with one embodiment;

FIG. 3C depicts another embodiment of the orthopedic alignment and balance system with GUI in accordance with one embodiment;

FIGS. 3D-3F illustrate extension gap measurement and range of motion during alignment and balance in accordance with one embodiment;

FIGS. 3G illustrates an integrated alignment and balance Graphical User Interface (GUI) in accordance with one embodiment;

FIG. 4A illustrates a sensorized distractor in accordance with one embodiment;

FIG. 4B is an exemplary method for sensorized distraction in accordance with one embodiment;

FIG. 5A illustrates an instrumented prosthetic device for prosthetic fit determination in accordance with one embodiment; and

FIG. 5B is an exemplary method for assessing prosthetic fit through range of motion in accordance with one embodiment;

FIG. 5C illustrates a prosthetic trial fit system in accordance with one embodiment;

FIG. 6A illustrates a mounting mechanism for providing rigid coupling to a receiver or transmitter in accordance with one embodiment; and

FIG. 6B illustrates a molded mounting mechanism for providing rigid coupling with a receiver or transmitter in accordance with one embodiment.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward.

In a first embodiment a cut-check system is provided to assess and report measurement of a bone cut. The measurement is provided with respect to an anatomical coordinate system defined according to a positioning of a sensorized mechanical plate with respect to one or more referenced anatomical landmarks. In one example, the cut-check system provides measurement of varus/valgus angle and anterior/posterior slope for distal femur cuts and proximal tibia cuts. The cut-check system permits a surgeon to check bone cuts made by mechanical jigs, guides or patient specific implants (PSI). It also provides distance measurements. A method of cut-check is also disclosed.

In a second embodiment a trial-check system is provided to assess trial insert sizing and kinematics with respect to fitted prosthetics. The trial insert is positioned between two prosthetic components and taken through a range of motion. Sensors mounted on the bones track movement through the range of motion. The trial-check device determines individual anatomical coordinate systems of a first bone and a second bone and reports an alignment there between. Measurement parameters include orientation, positioning and distance; used for assessing the trial insert, evaluating bone resection, extension gap dynamics and soft tissue release. A method of trial-check is also disclosed. In conjunction with cut-check, a coordinate system reference can be ported over with the method of trial-check to save time of one or more method steps.

In a third embodiment an integrated alignment and load balance system is provided to capture measurement information related to bone cuts and the forces thereto applied, for example, by the prosthetic components fitted onto the bone cuts. The system includes sensorized devices for evaluating cut angles and a load sensor inserted between bones for force measurement with respect to the cut angles. Orientation, positioning and distance are provided for evaluating bone resection, extension gap dynamics and soft tissue release.

In a fourth embodiment a sensorized distractor is provided to measure extension gap distance and as a locking mechanism for mounting sensorized devices thereon. The sensorized distractor includes a first fixed component and a second movable component that lock into position. It also serves as a brace for the trial-check system. Each component provides a visual geometric reference for positioning to anatomical landmarks. Once locked, each of the two components are modeled according to the locked position in view of the sensors on the distractor—a receiver on the first component, and a transmitter on the second component. In another arrangement, the distractor can provide calibration to the receiver and transmitter when mounted on the bones instead of on the distractor components.

In a fifth embodiment a prosthetic trial fit system is provided to assess and report prosthetic fit on one or more bone cuts. The prosthetics include a mounting mechanism for attaching sensors thereto, for example, a receiver or sensor. The system tracks the motion of the sensors on the prosthetics relative to one another and, with known information related to prosthetic three-dimensional (3D) models, determines the spatial relationship between the prosthetics. It reports prosthetic alignment and extension gap distances through a range of motion, including relative orientation.

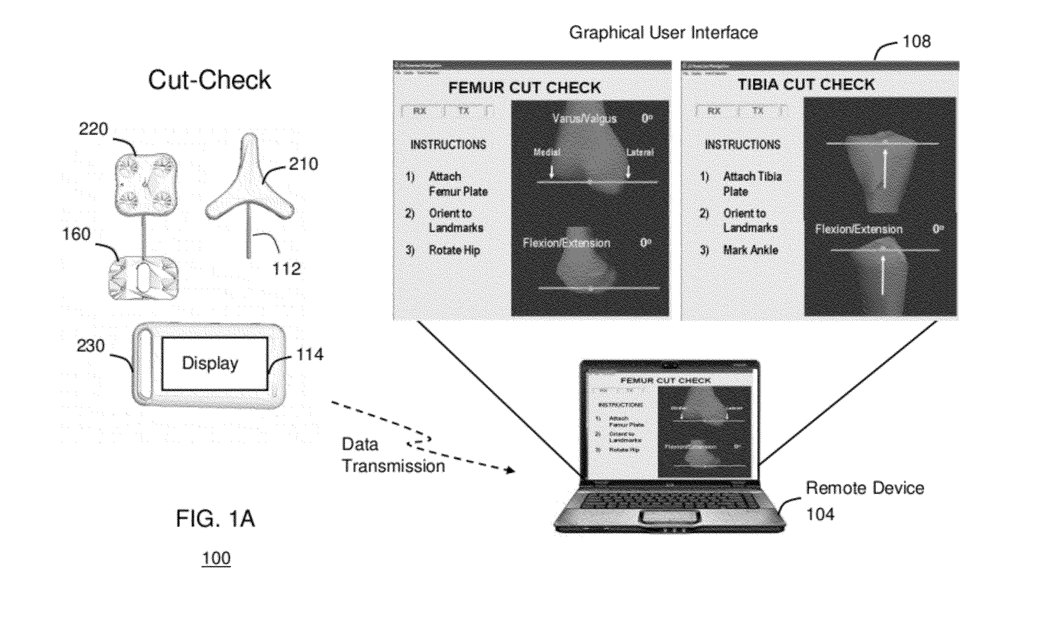

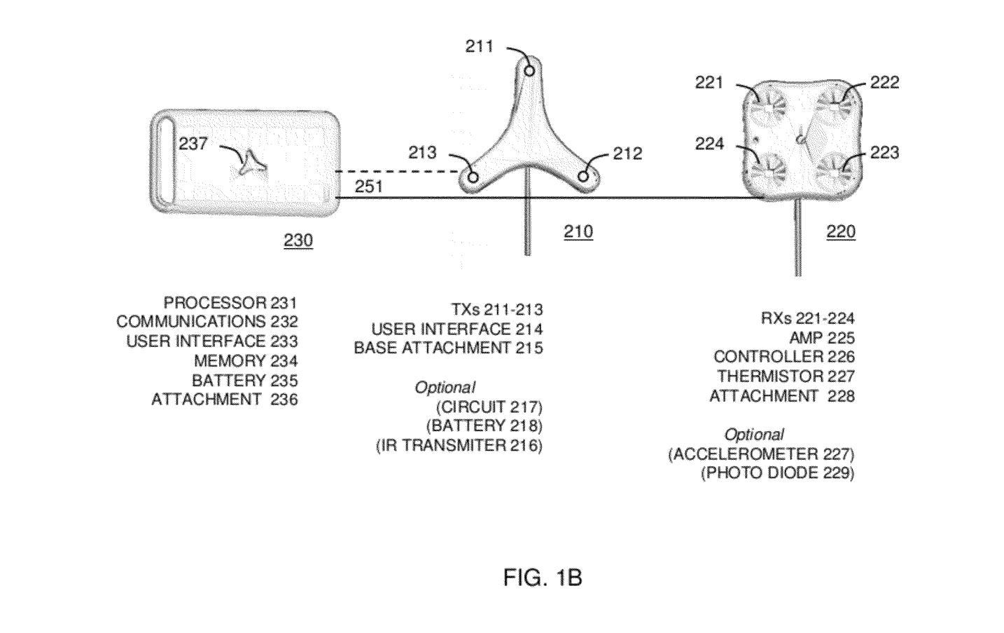

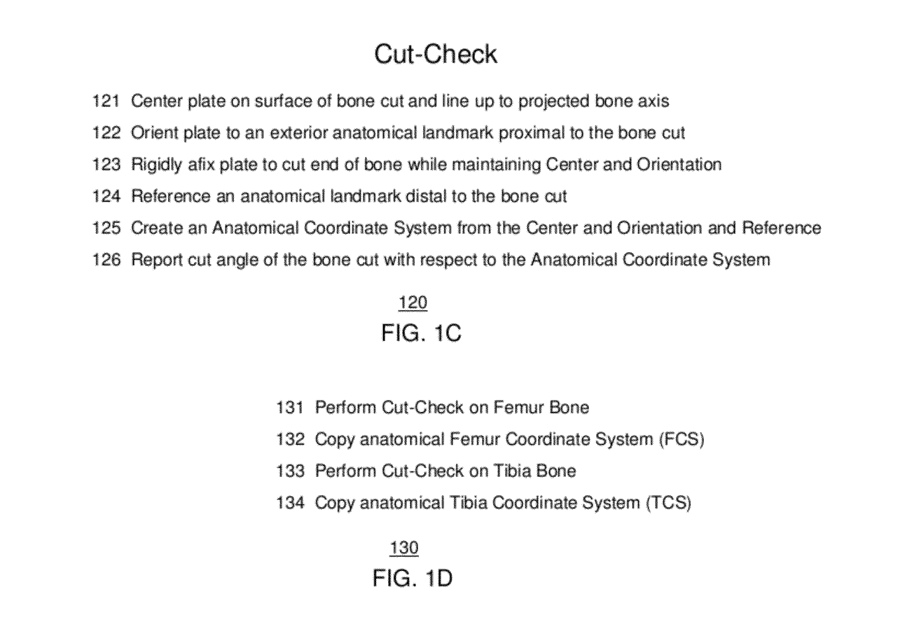

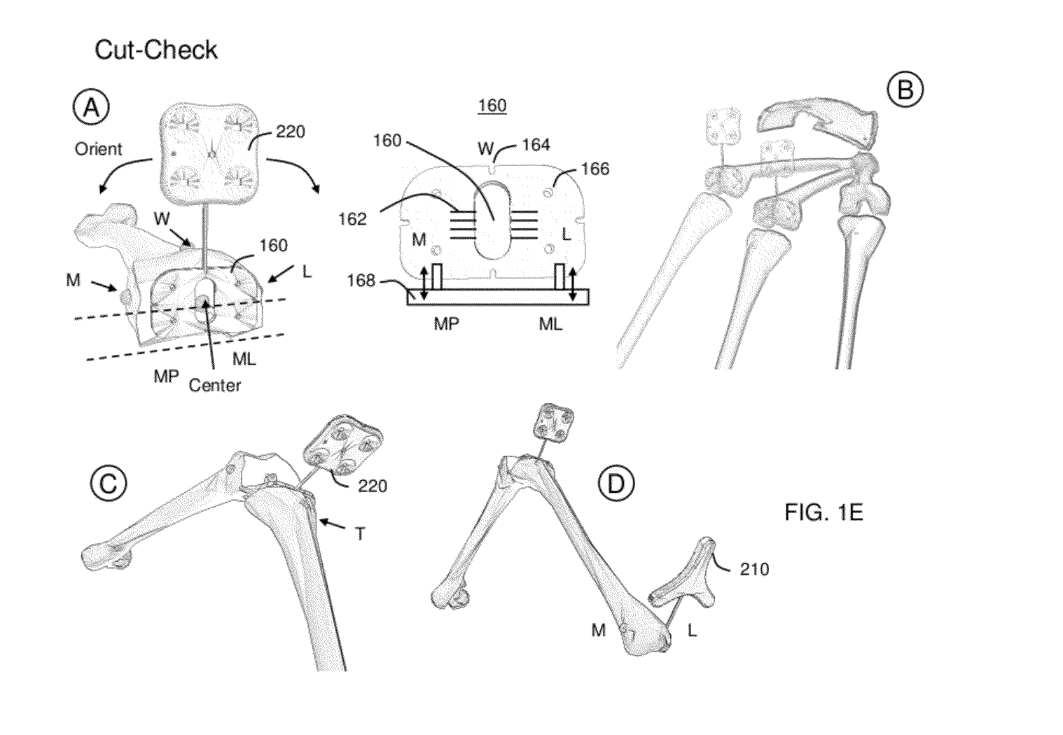

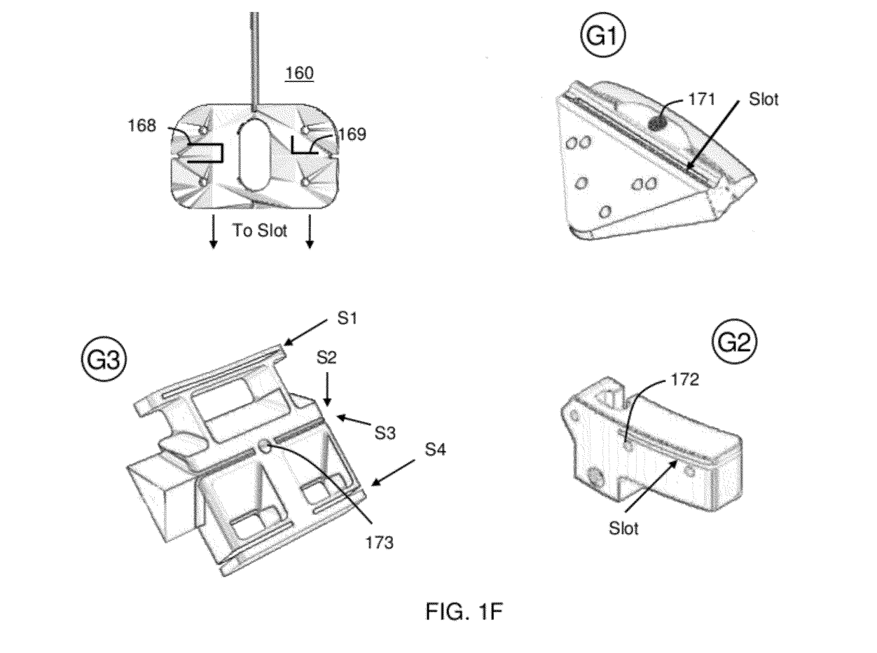

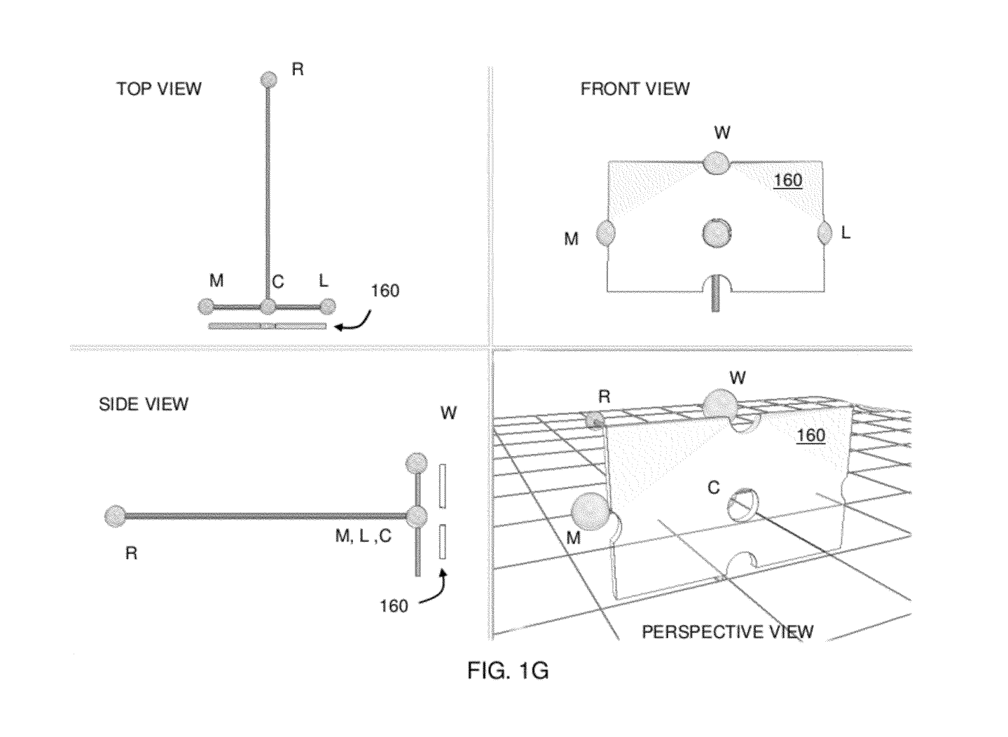

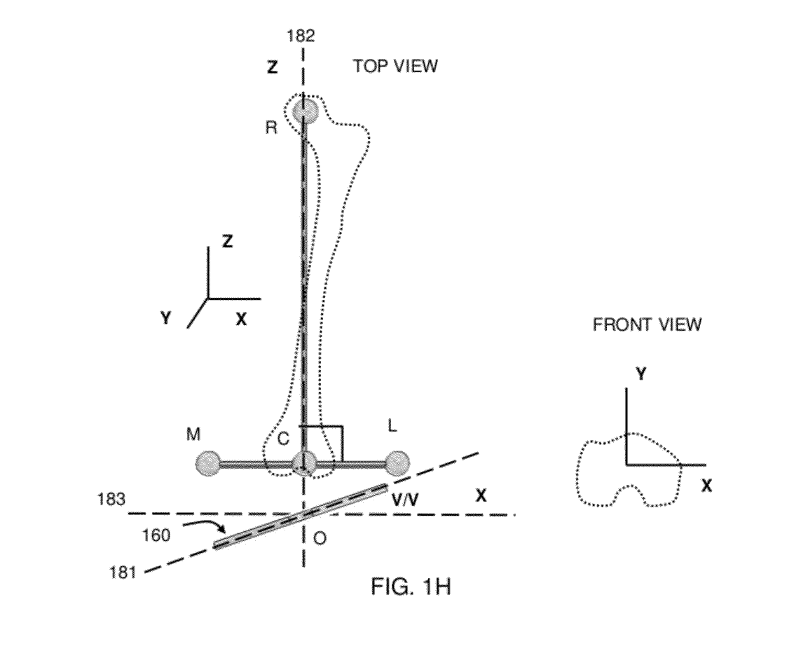

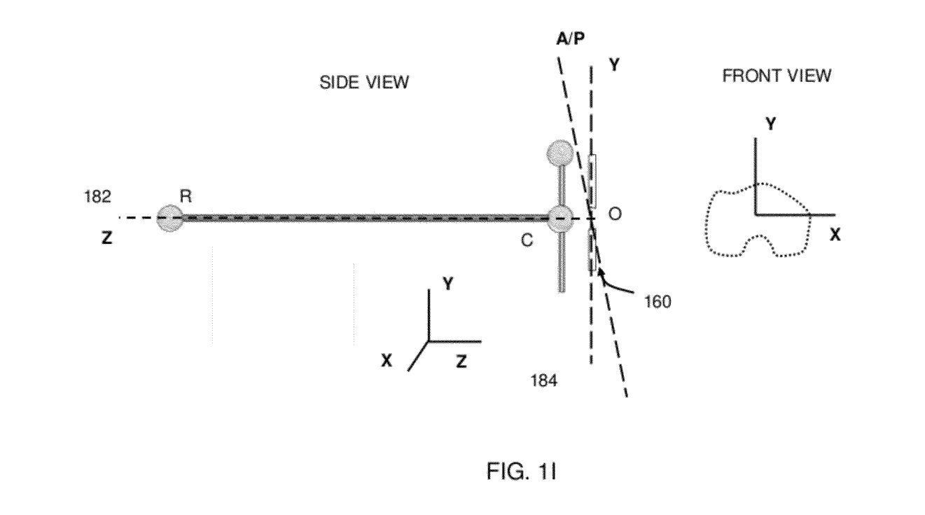

FIG. 1A depicts an exemplary embodiment of the cut-check system 100 for assessing bone cut angles. The system 100 includes a receiver 220 with an attachment mechanism to a plate 160, a transmitter 210 that transmits sensory signals to the receiver; and a pod 230 communicatively coupled to the receiver 220 and the transmitter 210. The pod 230 interprets the sensory signals and determines a position and orientation of the transmitter 210 with respect to the receiver 220. This permits the system 100 to report cut angle information when the plate 160 is properly positioned onto an exposed bone cut. In one arrangement the pod 230 includes a local display 114 mounted thereon for displaying positional information such as the cut angle. In another arrangement, the pod 230 can communicate the positional information to the remote device 104 which can display the information on a Graphical User Interface (GUI) 108 in a detailed format. The pod 230 is shown as a separate device although the internal electronics of the pod in other embodiments can be designed instead within the housing structure of the receiver 220.

FIG. 1B depicts exemplary components of the cut-check system 100 in accordance with one embodiment. As illustrated the system 100 comprises the pod 230, the transmitter 210 and the receiver 220. Not all the components shown are required; fewer components can be used depending on required functionality. The pod 230 can couple to the transmitter 210 and the receiver 220 over a wired connection 251 as shown. In another configuration the transmitter 210 is wireless to the pod 230 and receiver 220 as will be explained ahead. In the configuration shown, the pod 230 contains the primary electronics for performing the sensory processing of the sensory devices. The transmitter 210 and the receiver 220 contain few components for operation, which permits the sensory devices to be low-cost and light weight when mounted. In another configuration, the primary electronic components of the pod 230 are miniaturized onto the receiver 220 with the battery 235; thus removing the pod and permitting a completely wireless system.

The Transmitter 210 receives control information from the pod 230 over the wired connection 251 for transmitting sensory signals. In one embodiment, the transmitter 210 comprises three ultrasonic transmitters 211-213 for each transmitting signals (e.g., ultrasonic) through the air in response to the received control information. Material coverings for the transmitters 211-21 are transparent to sound (e.g., ultrasound) and light (e.g., infrared) yet impervious to biological material such as water, blood or tissue. In one arrangement, a clear plastic membrane (or mesh) is stretched taught. The transmitter 210 may contain more or less than the number of components shown; certain component functionalities may be shared as integrated devices. Additional ultrasonic sensors can be included to provide an over-determined system for three-dimensional sensing. The ultrasonic sensors can be MEMS microphones, receivers, ultrasonic transmitters or combination thereof. As one example, each ultrasonic transducer can perform separate transmit and receive functions.

The transmitter 210 also includes a user interface 218 (e.g., button) that receives user input for requesting positional information. In one arrangement, a multi-state press button can communicate directives to control or complement the user interface. It can be ergonomically located on a side to permit single handed use. The transmitter 210 may further include a haptic module with the user interface 214. As an example, the haptic module may change (increase/decrease) vibration to signal improper or proper operation. With the wired connection 251, the transmitter 210 receives amplified line drive signal's from the pod 230 to drive the transducers 211-213. The line drive signals pulse or continuously drive the transducers 211-212 to emit ultrasonic waveforms. In a wireless transmitter 210 configuration, the electronic circuit (or controller) 214 generates the driver signals to the three ultrasonic transmitters 211-213 and the battery 215 provide energy for operation (e.g., amplification, illumination, timing, etc). The IR transmitter 216 sends an optical synchronization pulse coinciding with an ultrasonic pulse transmission when used in wireless mode; that is, without line 251. A battery 218 can be provided for the wireless configuration when the line 251 is not available to provide power of control information from the pod 230. The communications port 216 relays the user input to the pod 230, for example, when the button of the interface 214 is pressed.

The Receiver 220 includes a plurality of microphones 221-224, an amplifier 225 and a controller 226. The microphones capture ultrasonic signals transmitted by the transducers 211-213 of the transmitter 210. The amplifier 225 amplifies the captured ultrasonic signals to improve the signal to noise ratio and dynamic range. The controller 226 can include discrete logic and other electronic circuits for performing various operations, including, analog to digital conversion, sample and hold, and communication functions with the pod 230. The captured, amplified ultrasonic signals are conveyed over the wired connection 251 to the pod 230 for processing, filtering and analysis. A thermistor 227 measures ambient air temperature for assessing propagation characteristics of acoustic waves when used in conjunction with a transmitter 210 configured with ultrasonic sensors. An optional photo-diode 229 may be present for supporting wireless communication with the transmitter 210 as will be explained ahead. An accelerometer 227 may also be present for determining relative orientation and movement. The accelerometer 227 can identify 3 and 6 axis tilt during motion and while stationary.

An attachment mechanism 228 permits attachment to the plate 160 (see FIG. 1) and other detachable accessories. As one example, the mechanism can be a magnetic assembly with a fixed insert (e.g., square post head) to permit temporary detachment. As another example, it can be a magnetic ball and joint socket with latched increments. As yet another example, it can be a screw post o pin to a screw. Other embodiments may permit sliding, translation, rotation, angling and lock-in attachment and release, and coupling to standard jigs or plates by way of existing notches, ridges or holes.

The Pod 230 comprises a processor 233, a communications unit 232, a user interface 233, a memory 234 and a battery 235. The processor 231 controls overall operation and communication between the transmitter 210 and the receiver 220, including digital signal processing of digital signals, communication control, synchronization, user interface functionality, temperature sensing, optical communication, power management, optimization algorithms, and other processor functions. The processor 231 supports transmitting of timing information including line drive signals to the transmitter 210, receiving of captured ultrasonic signals from the receiver 220, and signal processing for determination of positional information related to the orientation of the transmitter 210 to the receiver 220 for assessing and reporting cut angle information.

The processor 233 can utilize computing technologies such as a microprocessor (uP) and/or digital signal processor (DSP) with associated storage memory 108 such a Flash, ROM, RAM, SRAM, DRAM or other like technologies for controlling operations of the aforementioned components of the terminal device. The instructions may also reside, completely or at least partially, within other memory, and/or a processor during execution thereof by another processor or computer system.

The electronic circuitry of the processor 231 (or controller) can comprise one or more Application Specific Integrated Circuit (ASIC) chips or Field Programmable Gate Arrays (FPGAs), for example, specific to a core signal processing algorithm or control logic. The processor can be an embedded platform running one or more modules of an operating system (OS). In one arrangement, the storage memory 234 may store one or more sets of instructions (e.g., software) embodying any one or more of the methodologies or functions described herein.

The communications unit 232 can further include a transceiver that can support singly or in combination any number of wireless access technologies including without limitation Bluetooth, Wireless Fidelity (WiFi), ZigBee and/or other short or long range radio frequency communication protocols. This provides for wireless communication to a remote device 104 (see FIG. 1). An Input/Output port within the communications unit 232 permits portable exchange of information or data, for example, by way of Universal Serial Bus (USB).

The memory 234 stores received ultrasonic waveforms and processing output related to tracking of received ultrasonic waveforms and other timing information, state logic, power management operation and scheduling. The battery 235 powers the processor 231 and associated electronics thereon and also the transmitter 210 and the receiver 220 in the wired configuration.

The user interface 233 can include one or more buttons to permit handheld operation and use (e.g., on/off/reset button) and illumination elements 237 to provide visual feedback.

In a first arrangement, the receiver 220 is wired via a tethered electrical connection 251 to the transmitter 210. Timing information from the pod 230 tells the transmitter 210 when to transmit, and includes optional parameters that can be applied to pulse shaping. The processor 231 on the pod establishes Time of Flight measurements according to the timing with respect to a reference time base in the case of ultrasonic signaling. In a second arrangement, the receiver 220 is wirelessly coupled to the transmitter 210 via an optical signaling connection. The infrared transmitter 216 on the transmitter 210 transmits an infrared timing signal with each transmitted pulse shaped signal. The infrared timing signal is synchronized with the transmitting of the ultrasonic signals to the receiver 220. The receiver 220 can include the photo diode 229 which the pod 230 monitors to determine when the infrared timing signal is received. The pod 230 employs this infrared timing information to establish Time of Flight measurements with respect to a reference transmit time. The infrared transmitter and photo diode establish transmit-receive timing information to within microsecond accuracy.

For a single transmitter operation, the Receiver 220 senses ultrasonic waves transmitted by the Transmitter 210. The Receiver 220 determines positional information of the transmitter 210 from range and localization of received ultrasonic waves captured at the microphones. Notably, one or more transmitters 210 can be present for determining orientation among a group of transmitters 210. The pod 230 wirelessly transmits this information as positional data (i.e., translation vectors and rotational matrices) to the Display Unit 104. The Display Unit 104 processes the positional data to provide 3D visual rendering of alignment and orientation angles of the Transmitter 210 (and any devices thereto mounted, such as the plate 160). The Transmitter 210 intermittently transmits ultrasonic waves by way of the three (3) Transmitters. The transmission cycle varies over a 5-10 ms interval at each of the three transmitters; each transmitter takes turns transmitting an ultrasonic waveform. The ultrasonic waveforms propagate through the air and are sensed by the microphones on the Receiver 220. The Receiver 220 determines positional information of the Wand from range and localization of transmitted ultrasonic waveforms. The Receiver 220 measures the position and orientation of the Wand(s) in three-dimensions (3D) with respect to the Receiver 220 coordinate system.

Referring to FIG. 1C, a method 120 for cut-check is shown. The method 120 can be practiced with more or less than the number of steps shown. To describe the method 120, reference will be made to FIGS. 1E to 1F although it is understood that the method 120 can be implemented in any other suitable device or system using other suitable components. Moreover, the method 120 is not limited to the order in which the steps are listed in the method 120 In addition, the method 120 can contain a greater or a fewer number of steps than those shown.

The method 120 starts after one or more bone cuts have been made. The bone cut can be made with a standard mechanical cutting jig, guide, patient specific instrument (PSI) or other surgical instrument. At step 121, the plate 160 is centered on the surface of the bone cut and lined up to a projected bone axis. A projected bone axis is a projection of an axis along a surface. For instance, the projected axis of the femur bone that runs along the interior bone (or mechanical axis) along its long axis is a point, that when viewed down this axis corresponds to the bone center along the distal bone cut surface. Referring briefly to FIG. 1E, the plate 160 includes an open portion at the center to provide a visual reference for centering on the cut bone surface as shown in subplot A. The open portion of the plate 160 exposes visual notches 162 used for centering to the bone center. In practice, the anatomical bone cut center can be referenced from a hole created by an Intermedullary (IM) rod used with standard cutting jigs, indentation references from PSI jigs or through visual assessment of exposed anatomical landmarks.

At step 122, the plate 160 is then oriented to an exterior anatomical landmark proximal to the bone cut while the center is maintained. Distinguishing features of the plate 160 are provided to visually reference against the center of the bone cut, as noted above, and also for orientation reference to exterior anatomical landmarks. As one example, shown in subplot A, the M and L notches on the exterior sides of the plate 160 are visually aligned to the medial and lateral epicondyles on the femur bone. The surgeon having centered the plate 160 midway between the medial and lateral anatomical landmarks in the previous step orients the plate 160 to the M and L anatomical landmarks with the visual notches on the plate 160. For instance, the surgeon rotates the plate 160 in the plane of the bone cut at the center to visually align to the M and L landmarks when visually projecting down the projected bone axis; that is, the surgeon is “looking down” the long axis of the femur bone at the femur center of the bone cut. Other visual references on the plate 160 can also be employed for providing orientation reference. For example, Whiteside's line can be used for orientation reference. The notch W 164 is visually aligned to the anterior-posterior trochlear groove corresponding to Whiteside's line on the exposed femur bone. The plate 160 also provides an extendable posterior surface 168 for mechanically orienting to the medial and lateral posterior condyles of the femur bone. It is extendable up/down in 2 mm increments (see arrows). This surface 168 when centered to the femur center and then fit to conform to the M and L posterior condyles, in effect providing another means of anatomical landmark referencing.

At step 123, the plate is rigidly affixed to the cut end surface of bone while maintaining center and orientation established from the previous steps. As shown in FIG. 1E, the plate 160 provides mounting holes 166 for receiving two or more bone pins to hold the plate 160 firmly against the exposed bone cut. The mounting holes can be angled at various insertion angles, for example, about 2 degrees, to provide loaded fixation. The plate 160 is mounted by the means above when the cutting jigs, guides, PSI or instruments have been removed from the bone.

In the event, the surgeon desires to check the bone cuts while keeping the cutting jigs in place, the plate 160 is adaptable for insertion into the cut slots of those instruments. For example, referring to FIG. 1F, the plate 160 also serves to fit within a cutting jig slot of various cutting jigs as shown. The protrusions 169 are material excursions of the surface and slightly rounded outward to push up the plate material to provide a spring type for insertion fit. This provides an outward force when the plate 160 is inserted in the slot to keep the plate stationary. Other means are herein contemplated, for example, feature 168 which lifts out a rectangle portion. The plate 160 can insert into a femur cutting slot G1, a tibial cutting jig slot G2 and a 4 in 1 cutting block G3 to remain in place. The reference notches (162, 164, 168, M, L) on the plate 160 can also be used for centering (see 171-173) and orientation as previously noted.

Continuing method 120 at step 124, an anatomical landmark is referenced distal to the bone cut. This landmark is generally distal from the exposed bone cut and not directly accessible but can be determined by various means. For example, in the case of a femur bone cut, this anatomical landmark will be the femur head at the hip joint. In the case of a tibial bone cut, the anatomical landmark will be determined from exterior points on the ankle. The example of FIG. 1E provides illustration and is now further discussed. With reference to that figure, a work flow illustration for the cut-check system is provided for determining two different bone cuts: a distal femur bone cut angle (shown in subplots A to B), and a tibial bone cut angle (shown in subplots C to D).

The workflow steps for assessing the femur bone cut angle with the cut check system 100 is now presented. As shown in subplot A, and previously mentioned, the plate 160 is centered, oriented and fixed onto the distal femur cut in accordance with steps 121-123. In subplot B the distal anatomical landmark in step 124, corresponding to the femur head, is determined from a gentle rotation of the femur bone with respect to the stationary hip. In one arrangement, the receiver 220 that is coupled to the plate 160 is in line of sight of the transmitter 210 which is positioned within 1-2 meters thereto. Communication between the receiver 220 and transmitter 210 during the rotation (−20-30 seconds) provides for femur head identification. In another arrangement, the receiver 220 by way of the internal accelerometer determines a directional vector to the femur head during the rotation. This step effectively determines the femur head of the projected femur bone axis. Upon completion of the femur head identification the work flow steps for the femur bone cut check are complete.

The workflow steps for assessing a tibia bone cut angle with the cut check system 100 are now presented as another example of using the cut-check system 100. The workflow steps of the tibia bone cut angle are similar in principle to the femur bone cut. A small tibia center hole is made on the proximal bone cut for providing visual reference with a drill before start of the tibia cut check method 120. It is expected the hole will be expanded with a punch out in a following procedure to hold the tibial tray of a prosthetic knee. After the proximal tibia cut has been made, the plate 160 is visually centered over the small tibia center hole and oriented onto the tibial bone cut surface using visual notches on the plate 160 in accordance with step 121 of method 120. As shown in subplot C, the notch 164 of the plate 160 is then oriented (e.g., rotated in the cut plane) to the anatomical landmark corresponding to the tibia tubercle, T, and thereafter affixed in accordance with steps 122-123 of method 120. The transmitter 210 is then used to manually identify the medial and lateral malleolous (left and right ankle bone protrusions) on the ankle. This step effectively determines the ankle center of the projected tibia bone axis, in particular, a ratio of ˜60/40 along the line connecting the points. Upon completion of the ankle center identification the work flow steps for the femur bone cut check are complete.

For any bone cut, the steps 121-124 of centering and orienting the plate 160 and determining a distal landmark reference are done for creating an anatomical coordinate system. This corresponds to step 125 of method 102, where an Anatomical Coordinate System is created from the center, orientation and distal reference. Referring to FIG. 1G multiple view of a same exemplary Anatomical Coordinate System are shown (Top, Front, Side, and Perspective views). The notations M, C, L and W refer to Medial, Center, Lateral and Whiteside as previously noted during the orientation of the plate 160 along the bone surface. Notation, R, refers to the distal anatomical reference determined in step 124, for example, the femur head when the bone is the femur, or the ankle points when the bone is the tibia. The different views of FIG. 1G show that an orthogonal Anatomical Coordinate System (ACS) is created with proper centering and orientation of the plate 160 and identification of the distal reference R.

FIG. 1H illustrates the creation of the ACS from the anatomical points and involves the following steps. A first virtual line 181 is defined on the surface of the plate 160 between the M and L landmarks with its center at point O, what will be the ACS <x,y,z> origin. The angle of the plate 160 (shown at about 30 degrees up in the illustration) is irrelevant since the M and L virtual features are projected and aligned onto the surface of the bone cut with a center always at C. That is, the angle can be anywhere less than vertical. It is this alignment of the plate 160 to the anatomical landmarks that establishes the ACS orientation. A second virtual line 182 is defined from reference point R to O which creates the Z principal axis. Thereafter, a third virtual line 183 is uniquely created orthogonal to the second line 182 through the point O, which creates the X principal axis. Again, the angle of the plate 160 is irrelevant since this third line 183 is created orthogonal to the second line 182 at the origin, O. This inherently creates the XZ plane of the ACS. A fourth virtual line 184 (see FIG. 1I) is created orthogonal to the created XZ plane also at the point O which creates the Y principal axis. This creates the XYZ Anatomical Coordinate System with origin at point O.

Once the ACS is established in accordance with the method steps 121-124 above, the receiver 220 attached to the plate 160 reports it's own orientation relative to the ACS, which corresponds to step 126, wherein the cut-check system 100 reports a cut angle of the bone cut with respect to the Anatomical Coordinate System. Notably, the defined orientation of the ACS provides for projection of the plate 160 planar surface along the XZ plane for assessing varus/valgus (V/V) angle and along the YZ plane for assessing anterior/posterior angle (A/P). For instance, referring to FIG. 1H, the cut-check system evaluates the angle difference between line 181 and 183 to report the Varus/Valgus (V/V) angle with reference to rotation around the Y axis (see FIG. 1H) and the Anterior/Posterior (A/P) angle with reference to the X axis (see FIG. 1I). Notably, the alignment (and orientation) of the visual features (M and L, or W, or MP and ML) onto corresponding anatomical landmarks along the projected surface of the bone cut with reference to the common center, C, permits for creation of the virtual ACS. This also circumvents the need for manually registering these visual features at a particular spatial location, for example, using a wand tip to identify points.

Additional method steps are also herein contemplated for method 120, including generation of the ACS, wherein the transmitter 210 can be used to identify anatomical landmarks instead of orienting the plate 160. For instance, instead of rotating the plate 160 to align to the M and L landmarks, the transmitter 210 can be used with a wand tip to identify those points directly. A wand tip is placed on the transmitter and positioned at the appropriate landmarks. Similarly a probe rod attached to the transmitter can be used as a visual reference line with the trochlear groove, W. The rod with transmitter is for example vertically aligned with the grove and reported to the receiver 220 via a button press thereon. Similarly, the wand tip can be used to identify the medial and lateral posterior condyles instead of manually positioning the extension plate 168 (see FIG. 1E) on these anatomical landmarks. This permits for point identification of landmarks with the transmitter 210 rather than geometric positioning of the plate where certain landmarks are directly accessible and preferred, or in combination thereof, for a secondary confirmation to ensure visual orientation coincides directly with anatomical landmarks.

Referring back to FIG. 1D, a method 130 of cut-check to associate an Anatomical Coordinate System (ACS) with a bone is shown. Briefly, the method 130 is shown specific to workflow steps noted above in method 120 used during total knee replacement for measuring cut angles of the femur bone and the tibia bone. It is however not limited to leg bone cuts and can be practiced with more or less than the number of steps shown. Similar figures are referenced.

At step 131, a cut-check is performed on the femur bone. The method 120 of cut-check disclose above serves to report the cut angles (e.g., V/V and A/P) and to create the ACS. At step 132, the ACS is copied and saved as an anatomical Femur Coordinate System (FCS). More specifically, the coordinate framework including the origin, O, and the principal axes of the ACS are saved to memory.

At step 133, a cut-check is performed on the tibia bone. The method 120 of cut-check disclose above serves to report the cut angles (e.g., V/V and A/P) and to create the ACS. At step 134, the ACS is copied and saved as an anatomical Tibia Coordinate System (TCS). More specifically, the coordinate framework including the origin, O, and the principal axes of the ACS are saved to memory.

One advantage of method 130 is that the saved ACS for the femur or tibia can be recalled during another procedure, for example, during a “trial-check” without the need for identifying the distal anatomical landmarks (i.e., the femur head for the femur bone, and the ankle points for the tibia bone). That is, if the plate 160 is later properly centered and oriented on the bone cut in the exactly the same manner, the system can map the saved ACS (e.g., FCS or TCS) to the geometry of the plate 160 thereby recreating the ACS at the same location. This is because the plate 160 is itself a three-dimensional object that can be characterized as a reference model. As will be seen ahead, this saves time from re-registering points or capturing distal landmarks.

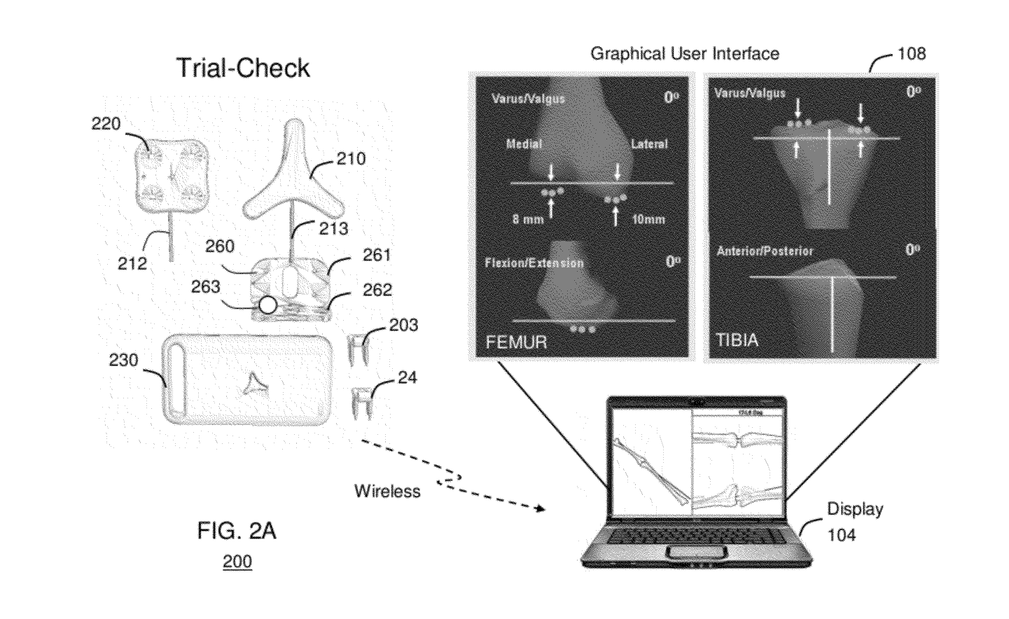

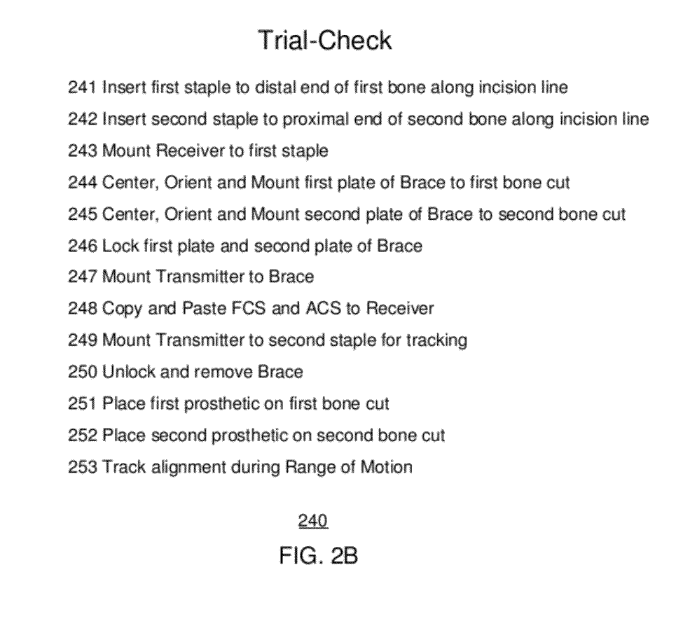

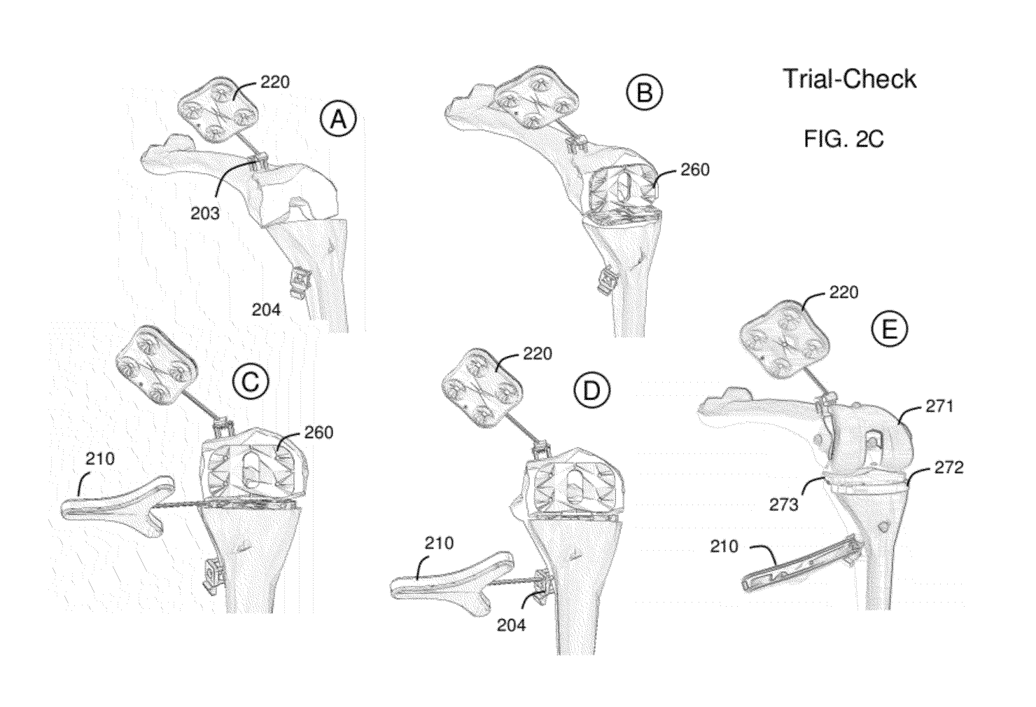

FIG. 2A depicts an exemplary embodiment of a trial-check system 200 for evaluating trial insert and prosthetic fit. The system 200 includes a receiver 220 with an attachment mechanism 212 to a first staple 203, a transmitter 210 that transmits sensory signals to the receiver with an attachment mechanism 213 to a Brace 260, and a pod 230 communicatively coupled to the receiver 220 and the transmitter 210. The attachment mechanism 213 of the transmitter 210 can also couple to a second staple 214. The Brace 260 includes a first plate 261, a second plate 262 and a lock 263. The two plates are free to move (or float) in relation to one another under constraint of an open lock. The lock 263 when closed (tightened) restricts motion between the first plate 261 and the second plate and holds the two plates at a confined orientation relative to one another. Each plate includes markings (or etchings) for visual reference to anatomical landmarks (e.g., see plate 160 in FIG. 1E). The plates on the Brace 260 can be each visually aligned to anatomical landmarks on a bone cut surface and affixed before being locked in place as part of a work flow procedure discussed ahead.

The pod 230 is communicatively coupled to the receiver 220 and the transmitter 210 and interprets the sensory signals and determines a position and orientation of these devices relative to one another. This permits the system 100 to assess bone cut orientations with respect to one another when the Brace 260 is properly positioned onto two exposed bone cuts. In one arrangement, the pod 230 can communicate the positional information to the remote device 104 which can display the information on a Graphical User Interface (GUI) 108 in a wider format. The pod 230 is shown as a separate device although the internal electronics of the pod in other embodiments can be designed instead within the housing structure of the receiver 220.

The cut-check system above assesses bone cuts. In the disclosed embodiment, the system comprises a receiver with an attachment mechanism to a plate, where the plate is oriented onto a surface of a bone cut; a transmitter that transmits sensory signals to the receiver to establish a base reference orientation; and a pod communicatively coupled to the receiver and the transmitter that interprets the sensory signals, determines a position and orientation of the plate with respect to the receiver, and from the orientation reports measurement of a varus and valgus angle and anterior and a posterior slope angle of the bone cut. The plate can be oriented to anatomical landmarks that map an anatomical coordinate system to the base reference orientation. The plate can also be oriented onto the surface of the bone cut and aligned to a medial-lateral axes to map a principal axes of the base reference coordinate system to an anatomical coordinate system reference. In one embodiment, plate slides into a slot of a patient specific instrument, and the pod reports an estimated bone cut angle of the patient specific instrument.

A method for cut-check is herein provided comprising the steps of centering a plate on a surface of bone cut and lining up to a bone axis, orienting the plate to an anatomical landmark proximal to the bone cut, affixing the plate to the bone cut while maintaining center and orientation, referencing an anatomical landmark distal to the bone cut, creating an anatomical coordinate system from the center, orientation and reference, and reporting a cut angle of the bone cut with respect to the anatomical coordinate system. The method includes determining a position and orientation of the plate with respect to a receiver, reporting a varus and valgus angle and anterior and a posterior slope angle of the bone cut from the orientation, and mapping a principal axes of a base reference coordinate system created by the receiver to the anatomical coordinate system. In one arrangement, as part of the method, the plate can be slid into a slot of a patient specific instrument to report an estimated bone cut angle of the patient specific instrument.

Referring to FIG. 2B, a method 240 for trial-check is shown. The method 240 can be practiced with more or less than the number of steps shown. To describe the method 240, reference will be made to FIGS. 1B, 2A and 2C although it is understood that the method 240 can be implemented in any other suitable device or system using other suitable components. Moreover, the method 240 is not limited to the order in which the steps are listed in the method 240. In addition, the method 240 can contain a greater or a fewer number of steps than those shown.

The method 240 starts after two bone cuts have been made on adjoining or opposing bones. As an example, the method 240 will be described in the context of a total knee replacement procedure for a femur bone and a tibia bone, although the method can apply to other adjoining bones (e.g., shoulder, hip, spine, etc.). The method can start in a state wherein the femur bone and tibia bone have been exposed, for example, after an incision has been made along the surface of the knee. Reference is made to FIG. 2C which illustrates the method 240 in an exemplary workflow for the total knee replacement procedure herein contemplated.

At step 241, the first staple 203 is inserted into distal end of femur bone along incision line. The sectioning of the incision sufficiently isolates the muscles, tendons and ligaments from the bone surface. It also provides direct contact with the distal end of the femur and the proximal end of the tibia for staple 203 insertion. Moreover, the placement of the staple 203 along the exposed bone minimizes pulling or tension on the staple 203 when the leg is bent during another work flow step ahead during range of motion. Subplot A of FIG. 2C illustrates an exemplary placement of the first staple 203 in accordance with method steps 241 and 242. At step 242, the second staple 204 is inserted to the proximal end of the tibia bone along the incision line. For the reasons just mentioned, the second staple 204 also provides rigidity with the bone through range of motion as it is does not rest or pull against soft tissue that could exert a shearing or pulling force during range of motion. The four prong staples 203-204 can also include an interior pin or screw support for further stability. Subplot A of FIG. 2C illustrates an exemplary placement of the second staple 204 in accordance with method steps 241 and 242. In certain cases, a single pin or screw instead of the four prong staple may provide adequate structural support.

At step 243, the receiver 220 is mounted to the first staple 203. The staple provides for coupling to the attachment mechanism 212 as shown in Subplot A of FIG. 2C. Briefly referring ahead to FIG. 7C, an exemplary mounting mechanism 600 is shown to provide variable orientation coupling with staple, sensor device (receiver 220 or transmitter 210), or bone. As illustrated, the screw portion 612 drills into the bone. The screw 612 has a ball 613 thereto mounted. The screw portion 612 can also be the staple 203; that is, four prongs instead of a single screw. The ball 613 fits within a housing portion 614 and may provide a tightening entry for a tool (e.g., Allan wrench slot); for example, for gripping or drilling. Accordingly, the housing portion 614 may include a slot 617 for inserting that tool (e.g., Allan wrench, screw driver, etc.). In another arrangement, the housing 614 may be decoupled prior to screw 612 insertion and then attached thereto. The cam lever 615 locks the housing 614 at a specific orientation for angling the mounting pin 616.

FIG. 7D shows an exemplary illustration where the receiver 220 with its attachment mechanism 212 couples to the housing portion 614 as an integrated mold. Alternatively, the housing portion 614 is molded with the housing of the Transmitter 201 or Receiver 220. In this case the housing 614 and attachment mechanism 212 are part of the Receiver 220. The mechanism design 600 permits rotational range spanning approximately 120 degrees.

Returning back to FIG. 2B at step 244, the first plate 261 of Brace 260 is mounted to the distal femur bone cut. It is centered and oriented in a similar fashion as the method 120 of cut-check system 100 and as previously explained above. That is, it is centered to the distal femur center, and then rotated so that the etches and distinguishing features on the plate 160 visually align with corresponding anatomical landmarks (e.g., M/L condyles, trochlear groove or M/L posterior condyles). The transmitter 210 can also be used as a secondary confirmation to register or mark these anatomical landmarks as previously described. The first plate 261 is then affixed to the distal femur cut while maintaining the center and orientation. This may be achieved by inserting up to 4 pins in the plate into the femur bone.

Once completed, the first plate 261 is rigidly coupled to the bone, and the second plate 262 is free to float, since the lock 263 is open. At step 245, the second plate 262 of Brace 260 is mounted to the proximal tibia bone cut. It is centered and oriented in a similar fashion as the method 120 of cut-check system 100 and as previously explained above. That is, it is centered to the proximal tibia center, and then rotated so that the etches and distinguishing features on the plate 160 visually align with corresponding anatomical landmarks (e.g., tibia tubercle, tibial plateau, M/L ankle points). The transmitter 210 can also be used as a secondary confirmation to register or mark these anatomical landmarks as previously described. The second plate 262 is then affixed to the proximal tibia cut while maintaining the center and orientation. This may be achieved by inserting up to 4 pins in the plate into the tibia bone.

At step 246, the lock 263 is tightened to constrain the first plate 261 and the second plate 262 of the brace 260 into a rigid position. This is illustrated in subplot B of FIG. 2C. The orientation of the plates is fixed upon locking, and which keeps the femur bone and the tibia bone in a fixed mechanical relationship; the bones are no longer free to move relative to one another. The lock 263 includes a tightening mechanism that secures the two plates into position. Since the plates are geometrically aligned and pinned to the bone cuts, they are permitted to move during locking if necessary. The locking ensures that the centering and orientation of the plates on the bone cuts is maintained and that the plates can no longer float after lock.

Once the Brace 260 is locked, the Transmitter 210 is temporarily mounted to the Brace 260 at step 247. If the Brace is locked into a predetermined configuration to establish the centering and planar orientation of the two plates relative to one another, than only a single mounting of the transmitter 210 is required. The Brace 260 contains various enumerated locking configurations each associated with a predetermined configuration. Once the transmitter 210 is coupled to the brace 260, the user then enters, or reports, which locking configuration (e.g., 1, 2, 3 or A, B, C etc.) of the brace 260 was manually selected. For example, the GUI 108 receives this as an entry parameter to determine the transformation (translation, scaling and rotation) between the two plates given their known geometries from the reported locking configuration. This single step then only requires a temporary mounting of the transmitter 210 to the brace 260 for reporting the configuration of the brace 260 to the mounted receiver 220. If however the lock is not closed in a predetermined configuration, then the transmitter 210 is separately attached in time to each of the plates at separate steps for registering the orientation of each plate with respect to the receiver 220.

At step 248, the coordinate systems of the first plate and the second plate are copied and pasted to the receiver 220. This is illustrated in subplot C of FIG. 2C. The ‘cut and paste’ terms are used to indicate that the anatomical coordinate system (ACS) captured previously in the cut-check method for each respective bone is recalled from device memory and reported to the receiver 220. The ACS for the femur is referred to as FCS, where the F is for femur. The ACS for the tibia is referred to as TCS, where the T is for tibia. Instead of requiring the user to repeat the cut check method 120 steps of creating an ACS for each bone, the transmitter 210 as coupled to the brace 260 only requires copying of the plate orientation. So, instead of requiring a femur head ID with trial-check to capture the distal femur reference point, the FCS model generated during cut-check is recalled thereby not requiring the user to capture the femur head ID. Similarly, instead of requiring ankle point indications with the transmitter 210 during trial-check, the TCS model generated during cut-check is recalled thereby not requiring the user to capture the ankle points again. This step is premised on the requirement that the first plate 261 and second plate 262 are mechanically attached during trial-check in an exact manner as when the corresponding plates were attached during cut-check.

As previously noted, the brace 260 is applied to lock the bones in a known configuration to one another. The receiver 220 is directly attached to the femur bone, and so the orientation of the first plate 261 thereto attached can be determined when the transmitter 210 is mounted to the first plate 261. That is, the receiver 220 determines, for example, by way of ultrasonic sensing, the orientation and location of the transmitter 210 through time of flight measurements of transmitted and received pulses from the plurality of transmit and microphone transducers. The pod 230 which acquires knowledge of this spatial transform also knows the plate geometries and their relationship to the receiver 220 and transmitter 210. That is, it contains three-dimensional (3D) models of the plates and mechanical attachments stored in memory. Accordingly, from the known mechanical couplings and models, the pod 230 can determine the mapping of the anatomical coordinate systems for each bone with respect to the sensor device locations.

With the brace 260 locked in position, a rigid mechanical coupling and model is established which allows the pod 230 to determine the relative location and orientation of the transmitter 210 with respect to the receive 220 whether it is marking points on the femur or tibia which in turns indicates the orientation and location of the plates thereto attached. Without the brace 260 however, the tibia would be free to move and its movement would need to be monitored, for example, by a second transmitter, else the receiver 220 would not be able to determine its location and orientation. In this case, the receiver 220 could track both the tibia via a secondary transmitter 210 and the orientation of the second plate 262 on the tibia cut with the first transmitter 210. However, this would require multiple transmitters that need to be tracked. By using the brace 260, the need for a second transmitter 210 is absent since it establishes a mechanical coupling between the femur and the tibia. The disclosure herein employs the brace methodology since it results in certain advantages over a dual transmitter approach.

Returning back to method 240 of FIG. 2B, upon completion of copy and paste the transmitter 210 is detached from the brace 260, and, at step 249 it is thereafter mounted to the second staple 204 for tracking. This is illustrated in subplot D of FIG. 2C. Once mounted to staple 204, a wand button thereon is pressed to report and confirm attachment, or a user entry to GUI 108 can be provided for same purpose. At step 250, the brace 260 is unlocked and the two plates are detached from the cut bone surfaces. This permits the bones to move freely in relation to one another. At step 251, the first prosthetic (femur prosthetic) is positioned onto the distal femur bone, and at step 252, the second (tibial tray prosthetic).

All bone cuts have been made at this time to ensure proper mounting of the prosthetics. Briefly referring to subplot E of FIG. 2C, an exemplary femur prosthetic 271 and a tibia tray prosthetic 272 are shown. A trial insert 273 is also inserted there between. These are the prosthetic devices mounted to the cut bones.

At this point, the trial-check 200 system can track the movement of the femur and tibia bone relative to each other as shown in step 253. More specifically, the pod 230 tracks the movement of the transmitter 210 attached to the tibia relative to the receiver 220 attached to the femur to assess alignment and orientation of the Femur Coordinate System relative to the Tibia Coordinate System with the prosthetic components in place. This also permits for determination of gap distance measurements and trial insert 273 sizing and kinematics between the tibia and the femur during range of motion as will be explained ahead.

The trial-check system assesses trial insert parameters. In one embodiment, it comprises a receiver that attaches to a first staple on a first bone within an incision line, a transmitter that attaches to a second staple on a second bone within the incision line, and a pod communicatively coupled to the receiver and the transmitter that interprets the sensory signals to determine a position and orientation of the transmitter with respect to the receiver and assesses an alignment of the first bone and the second bone. A trial insert can be positioned between two prosthetic components and taken through a range of motion, wherein the pod reports an applied force on the trial insert according to the alignment. It can also include a probe to capture anatomical landmarks on the first bone to create a first coordinate system and capture anatomical landmarks on the second bone to create a second coordinate system, wherein the pod reports the alignment with respect to orientation of the first and second coordinate system. In such arrangement, the pod reports measurement parameters including orientation, positioning and distance, and assesses forces on the trial insert, bone resection depth, extension gap dynamics and soft tissue release distances. In another configuration, the system includes a brace that attaches to the transmitter with a first plate having visual reference indications for orienting to the first bone; a second plate having visual reference indications for orienting to the second bone; and a mechanical coupler that permits a variable orientation of the first plate and second plate to one another and that locks and unlocks to a fixed orientation.

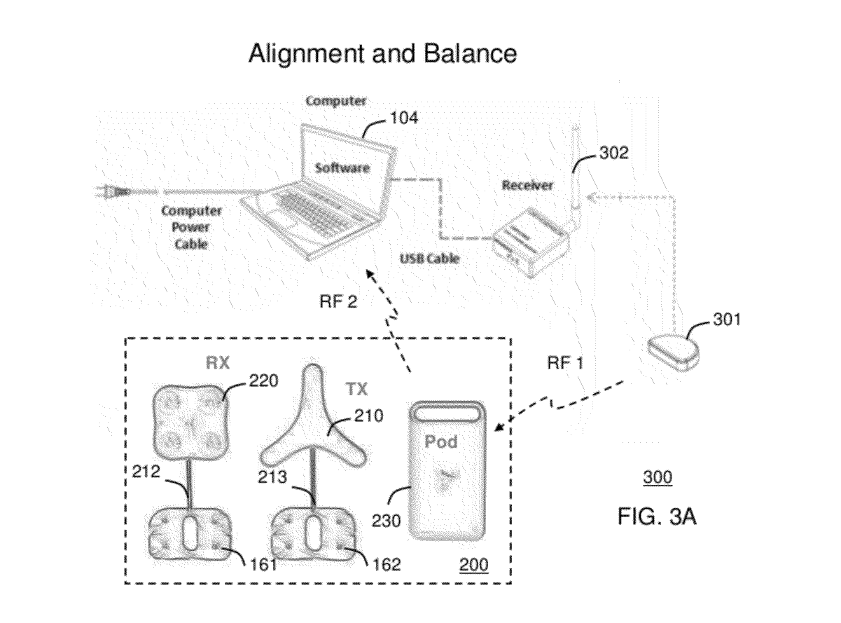

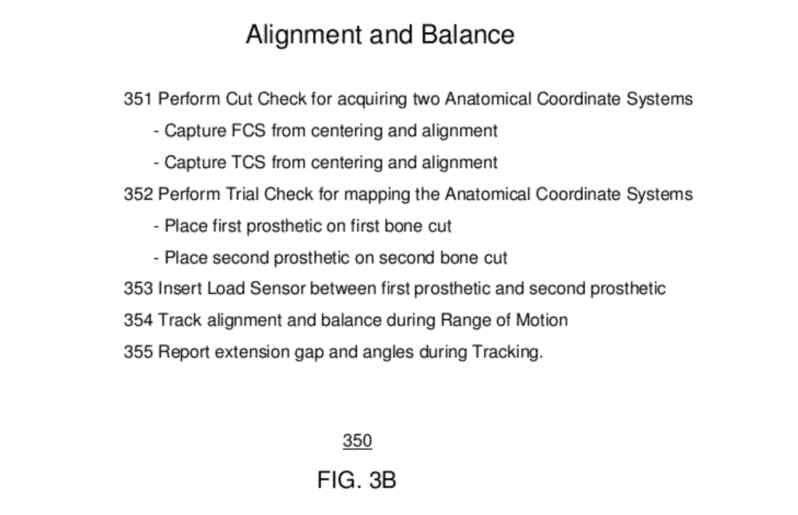

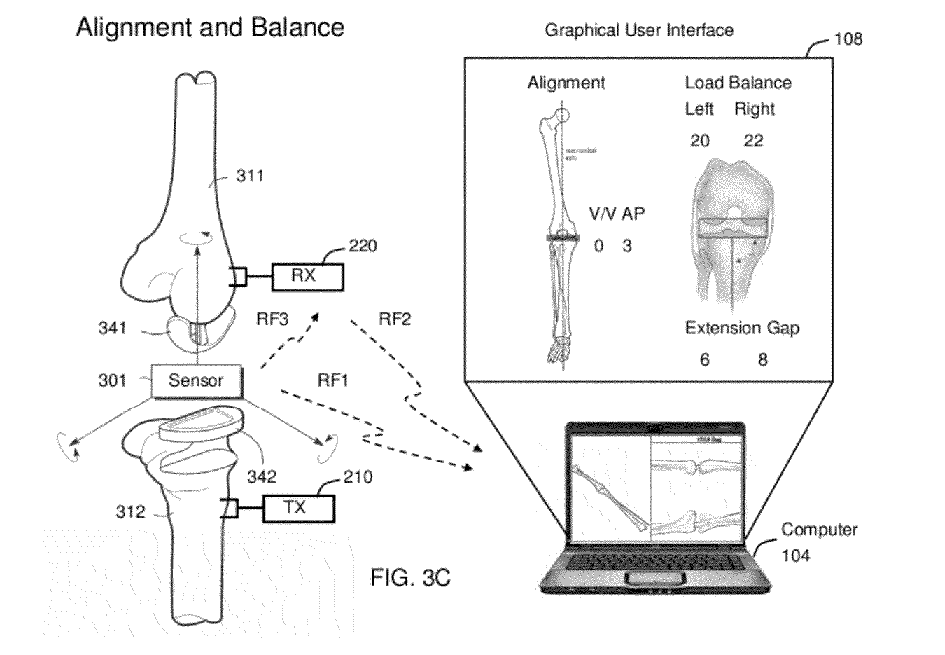

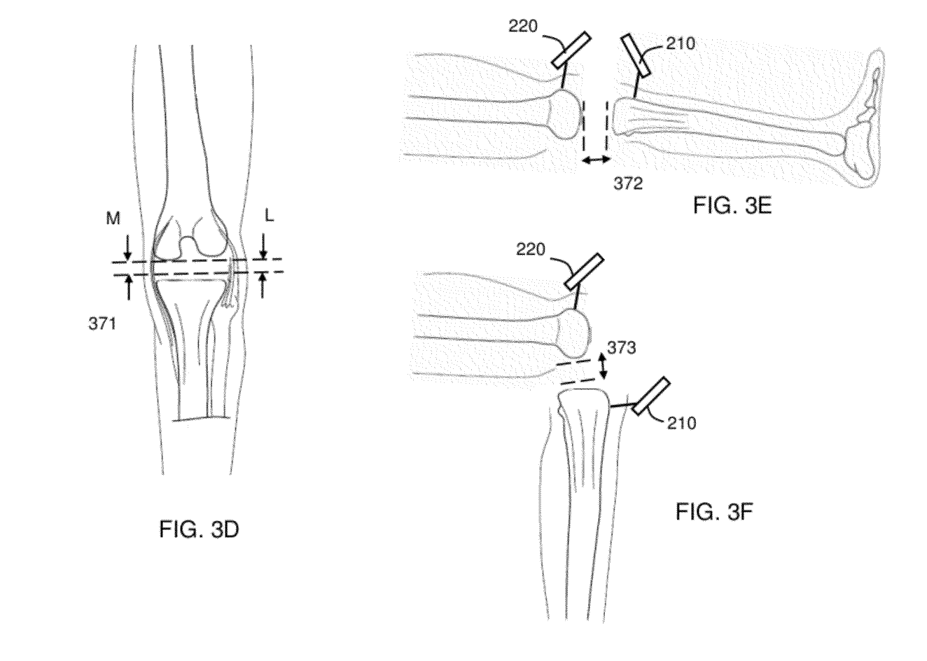

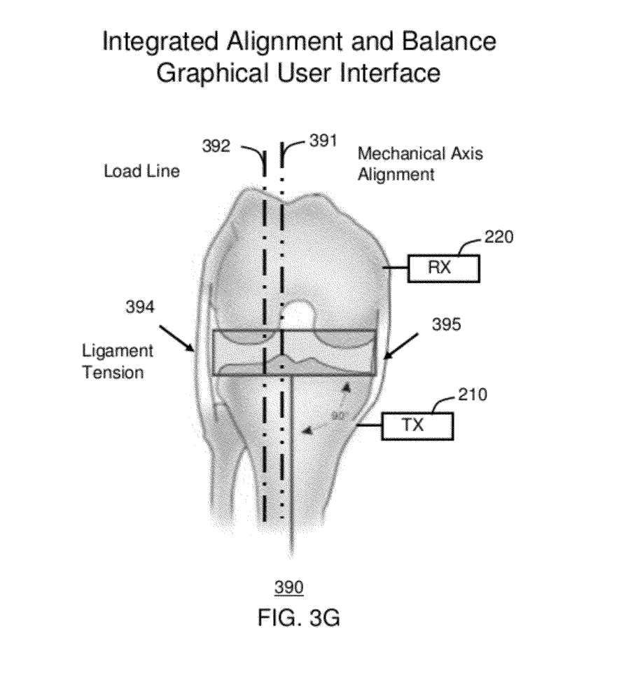

FIG. 3A depicts an exemplary embodiment of an alignment and balance system 300 to measure bone cuts and applied forces thereon, for example, after prosthetics are fitted onto the bone cuts and forces thereto coupled. The system 300 includes sensorized instruments of the trial-check system 200 for evaluating cut angles and a load sensor 301 inserted between bones with Receiver 302 for force measurement with respect to the cut angles. The system 300 provides orientation, positioning and distance measurements for evaluating bone resection, extension gap dynamics and soft tissue release.

The alignment and balance system 300 integrates the trial-check system 200 with the load sensor devices 301 and 302. The trial check system 200 includes the RX 220 with an attachment mechanism 212 to a first plate 161, the TX 210 to transmit sensory signals to the RX with an attachment mechanism 213 to a second plate 162, and the pod 230 communicatively coupled to the RX 220, the TX 210 and the computer 104. The Receiver 302 and the Load Sensor 301 are wirelessly attached in conjunction with the trial-check system 200 to the computer 104. The pod 230 is shown as a separate device although the internal electronics of the pod in other embodiments can be designed instead within the RX 220 or the Load Sensor 301.

The pod 230 can receive data communications from the load sensor 301 over channel RF1. It includes a communications unit with a transceiver configured to interpret communication data from the load sensor, for example, over a modulated radio frequency channel (e.g., GPSK, BPSK, PSK, etc.) The pod 230 includes internal electronics that also permit communication to the computer 104 in a wired or wireless mode (e.g., Bluetooth, Zigbee, RF, etc.) over communication channel RF2. In this manner, the pod 230 can act as a central hub to receive load force information from the load sensor 301 for balance data and transmit this information along with its own alignment data captured from the RX 220 and TX 210 to the computer 104 for processing, rendering and display. Alternatively, the pod 230 can also expose a display (e.g., touch screen) to present the information directly instead of relay to the computer 104. This may be advantageous for an integrated compact tool that visually provides alignment and balance information. In another configuration the Receiver 302 is coupled to the computer over a USB connection to provide data communication. As an example, the computer 104 receives from the receiver 302 load data applied onto the surface of the load sensor 301 direct through the USB connection instead of through the pod 230.

Referring to FIG. 3B, a method 350 for alignment and balance is shown. The method 350 can be practiced with more or less than the number of steps shown. To describe the method 350, reference will be made to FIGS. 3A-3G although it is understood that the method 350 can be implemented in any other suitable device or system using other suitable components. Moreover, the method 350 is not limited to the order in which the steps are listed in the method 350. In addition, the method 350 can contain a greater or a fewer number of steps than those shown.

The method 350 can start after bone cuts have been made on two bones in close proximity, for example, opposing or adjoining bones. The method 350 will be described in the context of a total knee replacement procedure as previously disclosed, with respect to a femur bone and a tibia bone, although the method can apply to other orthopedic procedures (e.g., shoulder, hip, spine, etc.). Reference is made to FIG. 3C which provides illustration for implementation of the alignment and balance system 300 and corresponding visualization on a GUI 108 during the total knee replacement procedure herein exemplified.

At step 351 a cut-check is performed for acquiring two Anatomical Coordinate Systems. The method 120 of cut-check previously presented can be relied upon here in part to achieve the cut-check registration for these two coordinate system references. Briefly recall that the method 120 of cut-check effectively creates a Femur Coordinate System (FCS) and a Tibia Coordinate System (TCS) by way of centering and orienting a plate 160 and with respect to a known three-dimensional models (i.e., plate object model saved in memory). Reference is also made to FIG. 1E which provides illustration for implementation of the cut-check system 100 and corresponding workflow during the total knee replacement procedure herein exemplified for such enablement.

At step 352, a trial-check is performed for mapping the two Anatomical Coordinate Systems. The method 240 of trial-check previously presented can be relied upon here in part to achieve the steps of recalling and storing (e.g., copy and paste) the FCS and TCS with respect to the femur and tibia bone relationship (e.g., model transformation) for tracking the femur bone relative to the tibia bone. Reference is also made to FIG. 2C which provides illustration for implementation of the trial-check system 200 and corresponding workflow during the total knee replacement procedure herein exemplified for such enablement. Recall that the method 240 of trial-check involves repositioning the plates onto the bones at identical locations previously performed during cut-check, and from a fixed relationship between the bones, as a result of the locking brace 260, establishes a transformation relationship between coordinate systems (e.g., FCS and TCS) that can be tracked between the RX 220 and the TX 210. The trial-check also includes the workflow steps of placing the first prosthetic on the first bone (component/femur) and the second prosthetic on the second bone (tray/tibia).

At step 353, the load sensor 301 is inserted between the first prosthetic (e.g., femur component) and the second prosthetic (e.g., tibia tray). For illustration, reference is now made to FIG. 3C which shows the RX 220 mounted onto the femur 311 above the femur prosthetic 341 component, the TX 210 mounted on the tibia 312 below the tibia tray prosthetic 342 component, and the load sensor 302 inserted between the femur prosthetic 341 and the tibia prosthetic 342. As previously noted, there are various communication path configurations (e.g., RF1, RF2 and RF3) for establishing integrated communication between the load sensor 301, the RX 220 and the computer 104 exposing the Graphical User Interface (GUI) 108.

At step 354, the system 300 tracks alignment and balance during range of motion. One example of a tracking system is disclosed in U.S. Pat. No. 7,724,355 and application Ser. No. 12/764,072 filed Apr. 20, 2010 the entire contents of which are hereby incorporated by reference. FIG. 3D-3F provides various illustrations to show range of motion and measurement of extension gap 371-373 during data acquisition of alignment and balance information. Range of motion refers to when the leg is moved between when it is in extension (straight) and when it is in flexion (bent). The range of motion can go between −10 degrees of hypertension to about 110 degrees of flexion, though it is a function anatomy and soft tissue.

At step 355, extension gap and angles are reported during the tracking. Assessing extension gaps during range of motion is performed after prosthetic placement as a clinical evaluation to examine the kinematics of motion due to the inserted prosthetics and the effect of bone cuts, trial insert sizing, and overall prosthetic fit. For instance, FIG. 3D illustrates gap distances between the medial (M) and lateral (L) compartments of the knee in extension. FIG. 3E-3F respectively illustrate the dynamics of extension gap distances during range of motion when the knee is in extension through flexion. The extension gap distances through the range of motion for each compartment can be presented on the GUI 108.

Referring to FIG. 3C, the GUI 108 shows an integrated display of alignment data (on the left) and balance data (on the right). As the example illustrates, alignment data provides varus/valgus (V/V) and anterior/posterior (A/P) information from the tracking of RX 220 and TX 210 with respect to the corresponding bones. Balance data provides indication of applied forces on the knee compartments, for example, a force (e.g., lbs/in², k/cm²) on each of the condyle knee surfaces (e.g., left side, right side). Although integrated at the user interface level, it can be appreciated that an efficient implementation integrates the software modules below the application level, that is, as code objects or modules that communicate with one another on a common programming platform.

Although the subplots are shown visually separate, an overlay GUI 390 as shown in FIG. 3G can be provided that combines the visual information for more efficiently displaying balance in view of alignment, for example, to show the mechanical axis 391 and the load line 392 with reference to anatomical load forces. Briefly, the RX 220 and TX 210 in conjunction with the pod 230 render of the mechanical axis 391; that is, the alignment between bone coordinate systems. The load sensor 301 and Receiver 302 render the load line 392; that is, the location and loading of the forces on the knee compartments that contribute to the overall stability of the prosthetic knee components. The overlay GUI 390 can also simulate soft tissue anatomical stresses associated with the alignment and balance information. For example, the GUI 390 can adjust a size and color of a graphical ligament object 391 corresponding to a soft tissue ligament 392 according to the reported alignment and balance information. The ligament object 391 can be emphasized red in size to show excess tension or stress as one example. Alternatively, ligament object 392 can be displayed neutral green if the knee compartment forces for alignment and balance result within an expected, or acceptable, range, as another example. Such predictive measurements of stress based on alignment and balance data can be obtained, or predetermined, from widespread clinical studies or from measurements made previously on the patient's knee, for example, during a clinical, or pre-op.

The integrated alignment and load balance system disclosed herein in one embodiment captures measurement information related to bone cuts and applied forces thereon, after prosthetics are fitted onto the bone cuts and thereto coupled, comprising sensorized devices for evaluating cut angles and a load sensor inserted there between bones for force measurement with respect to the cut angles, wherein orientation, positioning and distance are provided for evaluating bone resection, extension gap dynamics and soft tissue release. It can further include a distractor to measure extension gap distance, the distractor comprising a first component, a second component and a locking mechanism coupled thereto for mounting the sensorized devices thereon, wherein each of the first and second components provide a visual geometric reference for positioning to anatomical landmarks, and once locked, each of the first and second components are modeled according to the locked position in view of the sensorized devices on the distractor.

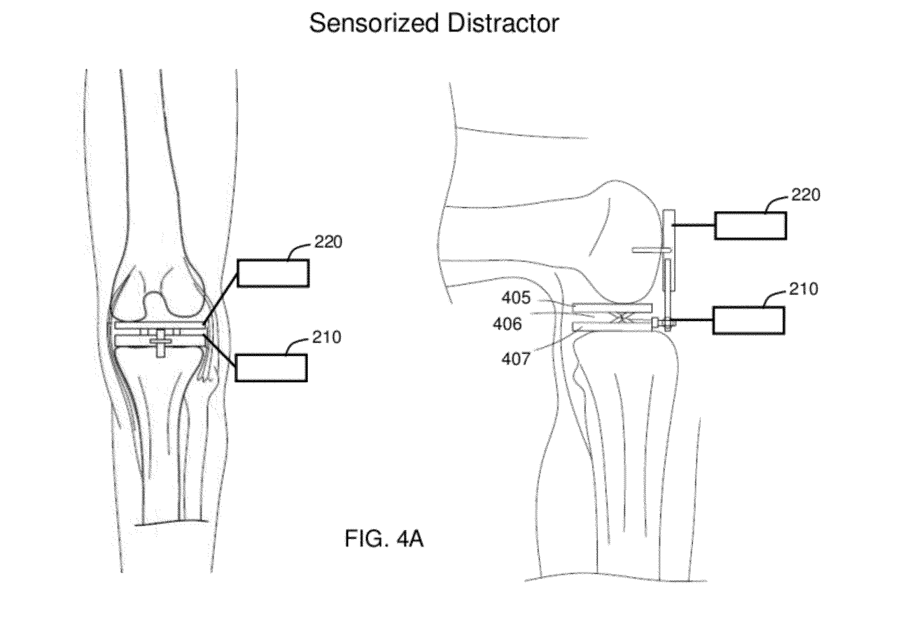

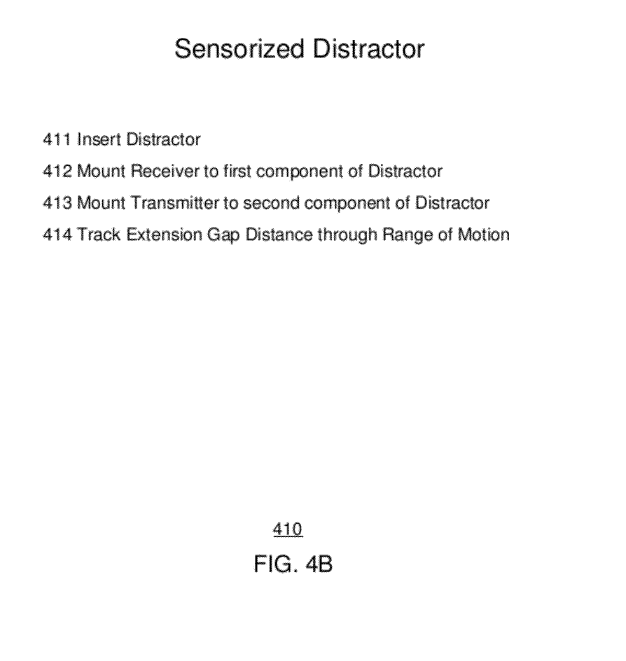

Referring to FIG. 4A, a sensorized distractor 400 is shown. The distractor 400 can serve as a locking mechanism for mounting sensorized devices thereon and for measuring extension gap distances. The sensorized distractor includes a first fixed component 405, a second movable component 407, and a mechanism 406 that moves the components relative to one another and into position. Each component provides a visual geometric reference for positioning to anatomical landmarks, similar to the cut-check plate 160. Once distracted, each of the two components are modeled according to a locked position in view of the sensors on the distractor—the receiver 220 on the first component 405, and the transmitter 210 on the second component 406. In another arrangement, the distractor can provide calibration to the receiver and transmitter when mounted on the bones instead of on the distractor components. One example of a distractor is disclosed in U.S. patent application Ser. No. 12/748,136 entitled “System and Method for Orthopedic Distraction and Cutting Block” filed Mar. 26, 2010 the entire contents of which are hereby incorporated by reference.

In one arrangement the sensorized distractor performs similar function to the brace 260 disclosed previously herein. That is, it provides for positioning of its top and bottom plates to a predetermined location on a bone cut surface. For example, the top plate of the distractor 400 is centered to the femur center landmark and oriented accordingly to femur anatomical landmarks (e.g., M/L, W, MP/LP). The bottom plate of the distractor 400 is centered to the proximal tibia center oriented accordingly to tibia anatomical landmarks (TT, MM/LM). The distractor is thereafter locked if used as a brace.

Referring to FIG. 4B, a method 410 for sensorized distraction is shown. The method 410 can be practiced with more or less than the number of steps shown. To describe the method 410, reference will be made to FIG. 4A although it is understood that the method 410 can be implemented in any other suitable device or system using other suitable components. Moreover, the method 410 is not limited to the order in which the steps are listed in the method 410. In addition, the method 410 can contain a greater or a fewer number of steps than those shown.

At step 411 the distractor 400 is inserted. Referring to FIG. 4B the distractor top plate 405 and bottom plate 406 are closed and inserted between the exposed bone cuts of the femur and tibia. This is usually done in flexion and within a spacing of between 10-20 mm; this generally corresponds to the amount of sectioned bone removed from the distal femur end and the proximal tibia. Once inserted, as step 412, the receiver 220 is mounted to the top plate 405. At step 413, the transmitter 210 is mounted to the bottom plate 406. At step 414, the Extension Gap Distance is tracked and reported through a Range of Motion, as shown in FIG. 4A. One example of a distractor is disclosed in U.S. patent application Ser. No. 12/748,112 entitled “System and Method for Soft Tissue Tensioning in Extension and Flexion” filed Mar. 26, 2010 the entire contents of which are hereby incorporated by reference.

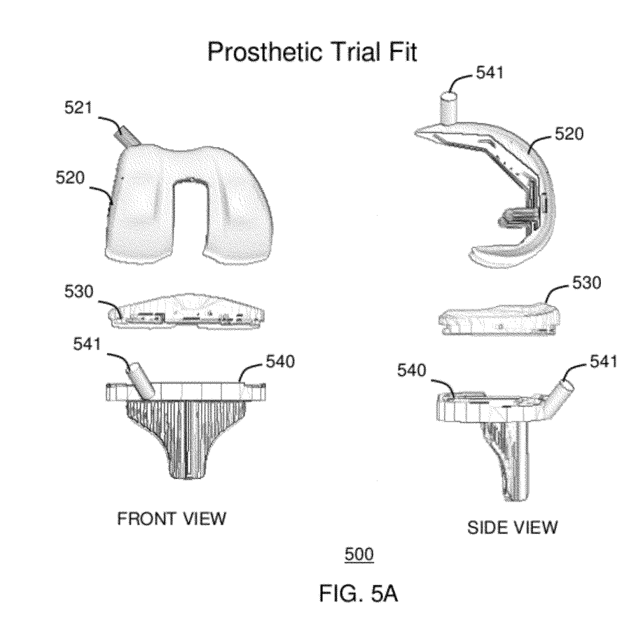

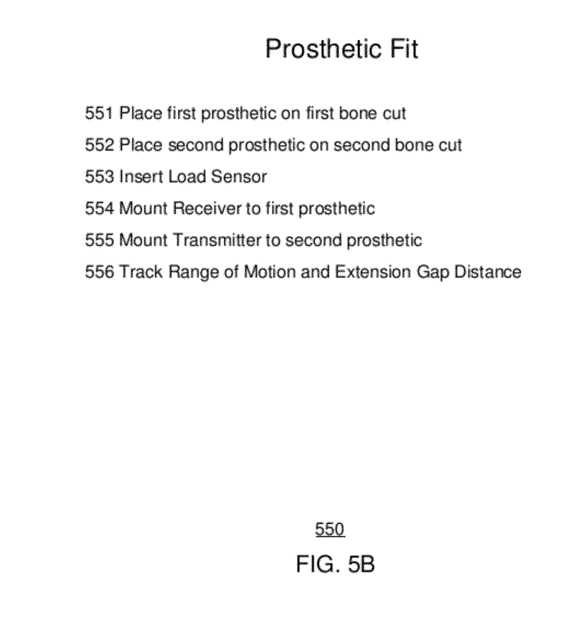

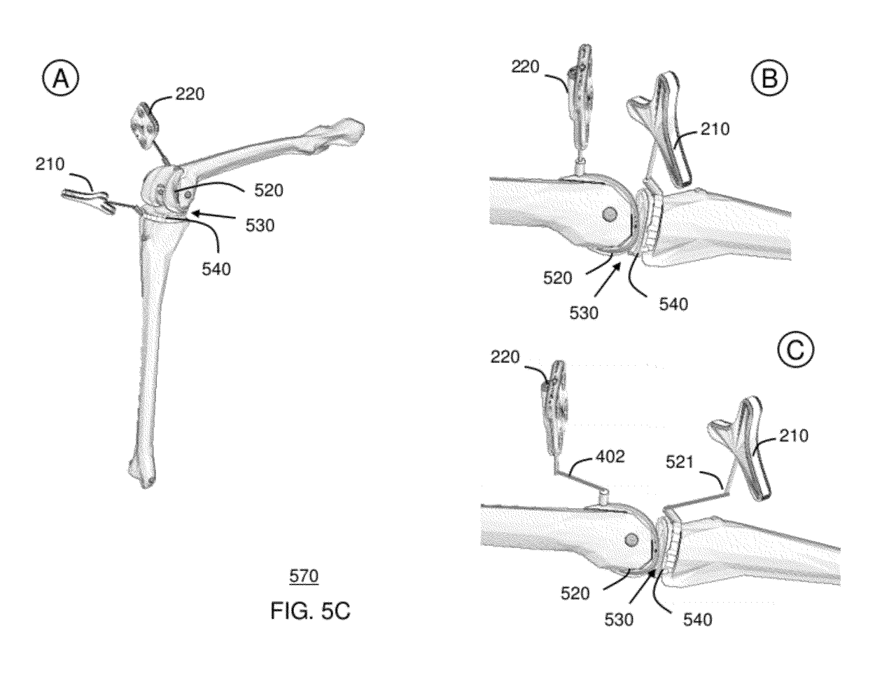

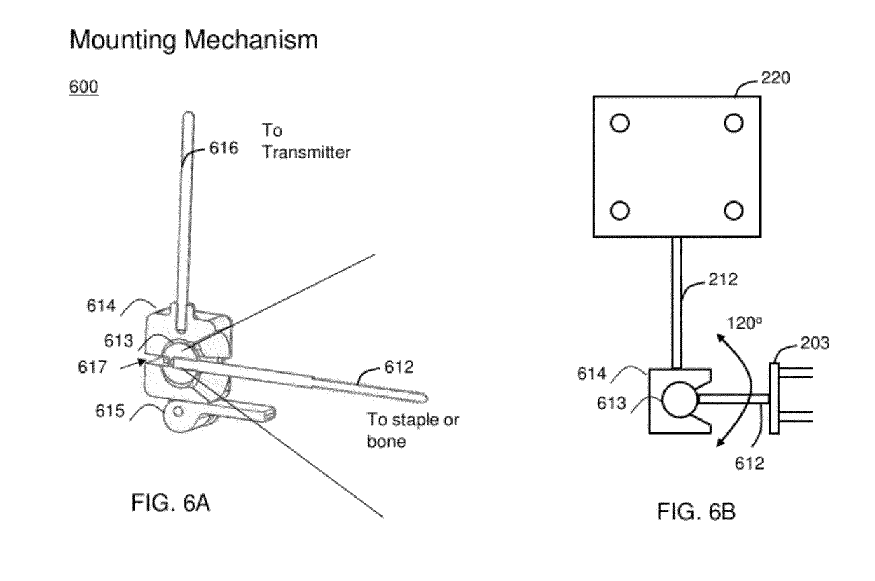

Referring to FIG. 5A, an instrumented prosthetic trial fit set 500 is provided to assess and report fit prosthetic fit with one or more bone cuts. The prosthetics are trial inserts in that they are sized and fitted only for temporary reference; a different prosthetic is used for the final prosthetic implant in a final step. The instrumented trial prosthetics 500 include a mounting mechanism for attaching sensors thereto, for example, a receiver or sensor. With sensors attached thereto, the prosthetic trial fit system tracks prosthetic motion relative to one another and, with known information related to the prosthetic three-dimensional (3D) models, determines the spatial relationship between the prosthetics and bones for reporting prosthetic fit. It also reports extension gap distances through a range of motion and relative orientation.

As illustrated, the femur component 520 includes a mounting mechanism 521 for attaching a sensor, such as the receiver 220 previously disclosed. The mounting mechanism can be designed (e.g. CAD) into the femur trial prosthetic model, for example, as a solid stainless steel piece. The tibia tray component 540 includes a mounting mechanism 541 for also attaching a sensor, such as the transmitter 210 previously disclosed. Similarly, the mounting mechanism can be designed into the tibia tray trial model, for example, as a solid stainless steel piece. The trial insert 530 can also include a mounting attachment (not shown) for receiving a sensor, such as the receiver 220 or transmitter 210. The attachment mechanisms 521/541 protrude at a location and angle for a generic patient anatomy to be least likely to interfere with normal leg motion. In particular, the attachment mechanisms 521/541 are sufficiently rotated off to the side so when Range of Motion is performed, and when the patella and patella tendon are repositioned over the knee cap, they minimally obstruct with the sliding of the patella over the prosthesis. Secondly, attachment mechanisms 521/541 are angled so as to provide the least amount of tension if temporarily placed under, or in proximity to, a tendon.

When the trial insert 530 is sensorized with a load sensor 520, and the prosthetic components are outfitted with the receiver 220 and transmitter 210, the prosthetic trial fit system provides balance and alignment information as related to expected prosthetic fit. That is, it assesses the prosthetic fit of the components with respect to one another as they are seated on the respective bone cuts. Whereas, the cut-check system 100 assesses cut checks from an anatomical coordinate system that is generated from bone anatomy, and the trial check 200 reports cut angle orientation with respect to load forces on bone cuts, the prosthetic fit system assesses the prosthetic fit of the prosthetic components on the bones. Aspects of the trial-check, such as using a wand tip to register anatomical points, can also be employed with prosthetic fit to report the prosthetic orientation on the bone anatomical structure.

An instrumented prosthetic trial fit system 570 as applied to the continuing knee example is shown FIG. 5C. Subplot A shows the receiver 220 is mounted to the femur trial prosthetic 520, and the transmitter 210 is mounted to the tibia tray prosthetic 540. They may be angled approximately 45 degrees to the side to permit line-of-sight viewing during range of motion, and to stay clear of patella motion. The trial insert or load sensor 530 is inserted between the femur and tibia bones within the knee joint. Subplot B shows a close distance of the sensors when a straight mounting rod is used. Subplot C shows that wider separation is achieved with a bent or “S” style mounting rod, which may be desired for tuning characteristics and to provide access to the exposed knee joint.

The prosthetic trial fit system 570 is unique from the trial-check system 200 in that the sensors are mounted directly to the prosthetic trials instead of on the bones. Accordingly, one distinguishing difference is that the coordinate systems of the prosthetic devices are tracked relative to one another rather than the anatomical coordinate systems of the bones. Although one expects the prosthetic component to precisely lay flush on the exposed bone cuts, this may not always be evident, or true. Due to variations in the cutting saw or surgical technique and patient anatomy, the prosthetic may not fit exactly in place on the bone every time. It also may be difficult to visualize or check. Accordingly, the prosthetic fit system 570 herein disclosed assesses the alignment of the prosthetics with respect to one another and also to the bone coordinate system using known instrumented prosthetic models. By way of the disclosed sensors, it determines anatomical mechanical axis, load line, load forces and corresponding prosthetic alignment and fit in extension, flexion and through range of motion.

In one embodiment, the prosthetic fit system assesses and reports prosthetic fit with one or more bone cuts fitted with prosthetics. The system includes a first prosthetic on a first bone with a first mounting mechanism for attaching a first sensor thereto, and a second prosthetic on a second bone with a second mounting mechanism for attaching a second sensor thereto. The system tracks the motion of the sensors on the prosthetics relative to one another and, with predetermined information related to three-dimensional (3D) models of the first prosthetic and second prosthetic, determines a spatial relationship between the first and second prosthetic for reporting prosthetic fit, relative orientation and extension gap distances through range of motion between the first and second prosthetic.

In one arrangement the prosthetic fit system further includes a tibia tray component that includes a mounting mechanism for attaching a sensor to track relative motion; and a load sensor for assessing applied forces between the first prosthetic and the second prosthetic. The pod is communicatively coupled to the first sensor on the first prosthetic and the second sensor on the second prosthetic, and is pre-programmed with a first prosthetic coordinate system of the first prosthetic device and a second prosthetic coordinate system of the second prosthetic device. The pod tracks relative motion of the first prosthetic coordinate system and the second prosthetic coordinate system to estimate alignment of a first anatomical coordinate system on the first bone and a second anatomical coordinate system on the second bone. The pod determines anatomical mechanical axis, load line, load forces and corresponding prosthetic alignment and fit in extension, flexion and through range of motion as previously noted.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived there from, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

These are but a few examples of embodiments and modifications that can be applied to the present disclosure without departing from the scope of the claims stated below. Accordingly, the reader is directed to the claims section for a fuller understanding of the breadth and scope of the present disclosure.

What is claimed is:

1. A cut-check system for assessing bone cuts, the system comprising:
a receiver with an attachment mechanism to a plate, where the plate is oriented onto a surface of a bone cut;
a transmitter that transmits sensory signals to the receiver to establish a base reference orientation; and
a pod communicatively coupled to the receiver and the transmitter that interprets the sensory signals, determines a position and orientation of the plate with respect to the receiver, and from the orientation reports measurement of a varus and valgus angle and anterior and a posterior slope angle of the bone cut.

2. The cut-check system of claim 2, wherein the plate is oriented to anatomical landmarks that map an anatomical coordinate system to the base reference orientation.

3. The cut-check system of claim 2, wherein the plate is oriented onto the surface of the bone cut and aligned to a medial-lateral axes to map a principal axes of the base reference coordinate system to an anatomical coordinate system reference.

4. The cut-check system of claim 1, wherein the plate slides into a slot of a patient specific instrument, and the pod reports an estimated bone cut angle of the patient specific instrument.

5. A method for cut-check comprising the steps of:
centering a plate on a surface of bone cut and lining up to a bone axis;
orienting the plate to an anatomical landmark proximal to the bone cut;
affixing the plate to the bone cut while maintaining center and orientation;
referencing an anatomical landmark distal to the bone cut;
creating an anatomical coordinate system from the center, orientation and reference; and
reporting a cut angle of the bone cut with respect to the anatomical coordinate system.

6. The method of cut-check in claim 5, further comprising:
determining a position and orientation of the plate with respect to a receiver, and
reporting a varus and valgus angle and anterior and a posterior slope angle of the bone cut from the orientation.

7. The method of cut-check in claim 6, further comprising:
mapping a principal axes of a base reference coordinate system created by the receiver to the anatomical coordinate system.

8. The method of cut-check in claim 6, further comprising sliding the plate into a slot of a patient specific instrument, and reporting an estimated bone cut angle of the patient specific instrument.

9. A trial-check system for assessing trial insert parameters, the device comprising:
a receiver that attaches to a first staple on a first bone within an incision line;
a transmitter that attaches to a second staple on a second bone within the incision line; and
a pod communicatively coupled to the receiver and the transmitter that interprets the sensory signals to determine a position and orientation of the transmitter with respect to the receiver and assesses an alignment of the first bone and the second bone.

10. The trial-check system of claim 9, further comprising a trial insert that is positioned between two prosthetic components and taken through a range of motion, wherein the pod reports an applied force on the trial insert according to the alignment.

11. The trial-check system of claim 9, further comprising a probe to capture anatomical landmarks on the first bone to create a first coordinate system and capture anatomical landmarks on the second bone to create a second coordinate system, wherein the pod reports the alignment with respect to orientation of the first and second coordinate system.

12. The trial-check system of claim 10, wherein the pod reports measurement parameters including orientation, positioning and distance, and assesses forces on the trial insert, bone resection depth, extension gap dynamics and soft tissue release distances.

13. The trial-check system of claim 10, further comprising a brace that attaches to the transmitter with
a first plate having visual reference indications for orienting to the first bone;
a second plate having visual reference indications for orienting to the second bone; and
a mechanical coupler that permits a variable orientation of the first plate and second plate to one another and that locks and unlocks to a fixed orientation.

14. An integrated alignment and load balance system to capture measurement information related to bone cuts and applied forces thereon, after prosthetics are fitted onto the bone cuts and thereto coupled, comprising sensorized devices for evaluating cut angles and a load sensor inserted there between bones for force measurement with respect to the cut angles, wherein orientation, positioning and distance are provided for evaluating bone resection, extension gap dynamics and soft tissue release.

15. The integrated alignment and load balance system of claim 14 further comprising a distractor to measure extension gap distance, the distractor comprising a first component, a second component and a locking mechanism coupled thereto for mounting the sensorized devices thereon, wherein each of the first and second components provide a visual geometric reference for positioning to anatomical landmarks, and once locked, each of the first and second components are modeled according to the locked position in view of the sensorized devices on the distractor.

16. A prosthetic fit system to assess and report prosthetic fit with one or more bone cuts fitted with prosthetics, the system comprising
a first prosthetic on a first bone with a first mounting mechanism for attaching a first sensor thereto,
a second prosthetic on a second bone with a second mounting mechanism for attaching a second sensor thereto,
wherein the system tracks the motion of the sensors on the prosthetics relative to one another and, with predetermined information related to three-dimensional (3D) models of the first prosthetic and second prosthetic, determines a spatial relationship between the first and second prosthetic for reporting prosthetic fit, relative orientation and extension gap distances through range of motion between the first and second prosthetic.

17. The prosthetic fit system of claim 16, further comprising:
a tibia tray component that includes a mounting mechanism for attaching a sensor to track relative motion; and
a load sensor for assessing applied forces between the first prosthetic and the second prosthetic.

18. The prosthetic fit system of claim 16, further comprising:
a pod communicatively coupled to the first sensor on the first prosthetic and the second sensor on the second prosthetic, where the pod is pre-programmed with a first prosthetic coordinate system of the first prosthetic device and a second prosthetic coordinate system of the second prosthetic device.

19. The prosthetic fit system of claim 18, wherein the pod tracks relative motion of the first prosthetic coordinate system and the second prosthetic coordinate system to estimate alignment of a first anatomical coordinate system on the first bone and a second anatomical coordinate system on the second bone.

20. The prosthetic fit system of claim 18, wherein the pod determines anatomical mechanical axis, load line, load forces and corresponding prosthetic alignment and fit in extension, flexion and through range of motion.
